(19) United States
(12) Patent Application Publication
(10) Pub. No.: US 2009/0252697 A1
(43) Pub. Date: Oct. 8, 2009

(54) METHOD FOR PERMANENTLY RESHAPING KERATIN FIBERS WITH AN ACID PRE-TREATING COMPOSITION

(76) Inventors: Philippe BARBARAT, Bois-Colombes (FR); Stefania NUZZO, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/420,423

(22) Filed: Apr. 8, 2009

(30) Foreign Application Priority Data
Apr. 8, 2008 (FR) 08/52355

(51) Int. Cl. A61K 8/36; A61K 8/30; A61K 8/46; A61Q 5/04

(52) U.S. Cl. 424/70.5; 424/70.2

(57) ABSTRACT

A method for permanently reshaping keratin fibers, such as hair, comprising applying onto the keratin fibers a pre-treating composition comprising, in a cosmetically acceptable medium, at least one entity chosen from non reducing inorganic acids, non reducing organic acids, and salts thereof, wherein the pH value of said pre-treating composition ranges from 2 to 5; rinsing the keratin fibers; applying onto the keratin fibers a treating composition comprising, in a cosmetically acceptable medium, at least one hydroxide compound; and reshaping the keratin fibers, wherein the reshaping occurs after applying the pre-treating composition and at any time before, during, or after applying the treating composition.

This application claims benefit of priority under 35 U.S.C. § 119 to French Patent Application No. FR 08/52355, filed Apr. 8, 2008, the contents of which are also incorporated herein by reference.

The present disclosure relates to a method for permanently reshaping the keratin fibers, such as for straightening the same, comprising applying an acid-containing composition and applying a hydroxide compound-based composition.

Two methods are typically used for permanently reshaping the hair, both based on the breaking of the S—S disulfide bonds that are present in keratin (cystine).

The first method for permanently reshaping the hair in a first step typically opens the disulfide bonds with a composition comprising a reducing agent (reducing step), then, once the hair has been for example rinsed off, reforms in a second step said disulfide bonds, by applying onto the hair, which has been placed beforehand under tension with curlers or equivalent, or otherwise set in form or straightened, an oxidizing composition or a so called fixative (oxidizing step), so as to give the hair the expected shape. This method thus can make it possible to either wave the hair, or to shape, decurl, or straighten the hair.

The second method for permanently reshaping the hair typically conducts a so-called lanthionization operation via a composition comprising a base belonging to the hydroxide family. This leads to replacement of the disulfide bonds (—CH₂—S—S—CH₂—) with lanthionine bonds (—CH₂—S—CH₂—). This lanthionization operation involves two consecutive chemical reactions:
The first reaction comprises a beta-elimination on the cystine brought about by a hydroxide ion, leading to the breaking of such bond and to the formation of dehydroalanine, as illustrated on the following reaction scheme.

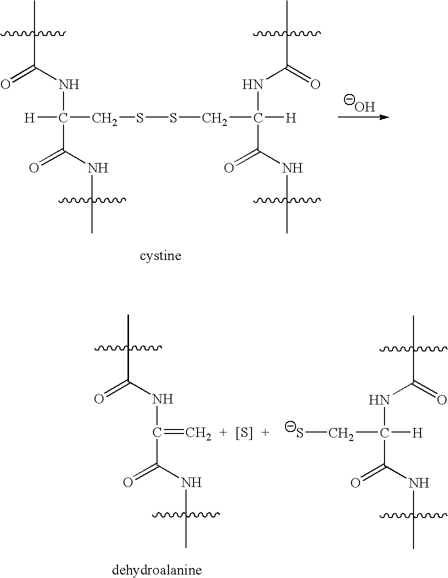

The second reaction comprises the reaction of the dehydroalanine with a thiol group. Indeed, the double bond of the dehydroalanine formed is a reactive double bond. It may react with the thiol group of the cysteine residue that has been released to form a new bond, referred to as a lanthionine bridge or bond or residue. This second reaction is illustrated on the following reaction scheme.

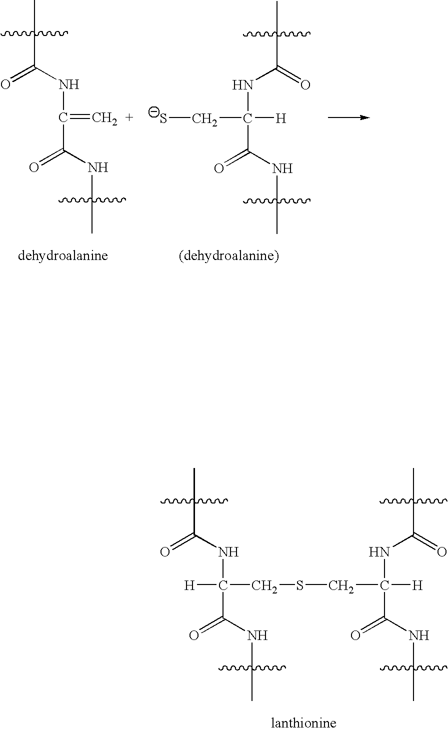

As compared to the previously described first method that uses a reducing agent, this lanthionization method does not require any fixing step, since the formation of the lanthionine bridges is irreversible. It thus comprises a single step and can make it possible to either wave the hair or to shape, decurl, or straighten the same. However, it is mainly used to set in form naturally frizzy hair.

Generally speaking, the reshaping effect durability provided by the thiols and sulfites by reducing the disulfides, and thereafter by fixing, can be much shorter than that possibly obtained via the lanthionization method.

The compositions that are usually used for conducting the lanthionization process comprise a hydroxide as a base, such as sodium hydroxide, guanidinium hydroxide, and lithium hydroxide. Sodium hydroxide and guanidinium hydroxide are the two most frequently used agents for setting in form or decurling naturally frizzy hair. They can possess a plurality of benefits as compared to ammonium thioglycolate and sulfites, such as they do not release any unpleasant smell, they just require a single step to be carried out for reshaping the hair (and thus may have a shorter treatment duration), and they may provide a more efficient way to reshape the hair on a much longer time basis.

However, those hydroxides that are used without any acid pre-treatment may, as a major drawback, suffer from being caustic.

Such causticity can affect the scalp by causing irritations that may be sometimes severe. It is known to partly counteract this inconvenience by previously applying onto the scalp a greasy-texture barrier cream, often called a “base” or “a base cream”, where the word “base” as used herein does not have the chemical meaning of basic agent. When such barrier cream is combined with hydroxide in a single composition, it is generally called “no-base”, as opposed to the hereabove given name. It is generally preferred to use such “no-base” method.

The hydroxide causticity may also negatively affect the hair condition by firstly possibly rendering it rough to the touch, and secondly possibly making it much more brittle, sometimes even so brittle to the point of degrading or even of dissolving the hair if the treatment is too long. Hydroxides also sometimes cause the hair natural color to bleach.

In addition, hydroxides are known to be good agents for hydrolyzing amide functional groups (see for example March's Advanced Organic Chemistry, 5ed., Wiley Interscience, New York, “Hydrolysis of Amides” pages 474 and ff.) which therefore lead to the peptide bond break through direct nucleophilic attack. Without wishing to be bound by any theory, the damage to the hair, and in a broad sense to the keratinic materials, that could be observed are thought to be mainly due to the partial hydrolysis of the amide bonds in keratin.

Therefore, known straightening treatments may seriously impair the hair condition. Those impairments result from the fact that the intrinsic properties of the hair may have been physically, chemically, and physico-chemically strongly modified. Those properties can include the diffusion properties upon which the swelling and the porosity of the hair depend for instance. Alkaline straightening treatments (comprising soda or the same) are known as being capable of strongly impairing the diffusion properties within the hair.

There is therefore a real need in hair reshaping for compositions that would be less aggressive to the hair.

Acid-based treatments are known as post-treatments for improving the hair quality following a cosmetic treatment such as bleaching, perming, and straightening.

Moreover, a free radical-scavenger composition is known from WO 01/06997 for pre-treating the hair, which has to be applied before a chemical treatment, such as for hair curling or smoothing. The pre-treating composition comprises ascorbic acid. However the reference does not disclose soda-based permanent reshaping of the hair.

It is also known from U.S. Pat. No. 4,709,712 to use a composition in the form of an aqueous gel comprising a polycarboxylic acid polymer to protect the scalp from the caustic solution that may run down during the hair treatment, such as during a perm procedure. But there is no mention of a permanent reshaping using soda (lanthionization).

A keratin fiber lanthionization method is also known from WO 02/085317, consisting in applying a composition as a pre-treatment comprising at least one organic nucleophile for increasing the tensile strength of the keratin fibers, thereafter in applying a relaxing composition to enable the lanthionization. The organic nucleophile may be for example a basic amino acid derivative.

A keratin fiber lanthionization method is also known from WO 03/015731, consisting in applying a pre-treating composition comprising at least one reducing agent, then in applying a composition comprising a hydroxide ion generating agent to carry out the lanthionization.

The present disclosure therefore can provide a method for permanently reshaping keratin fibers, for example a method for straightening keratin fibers, which improves the fiber quality, for instance hair smoothness, shine, and/or feeling, which avoids at least one of the drawbacks mentioned above, such as, restraining the impairment of the fiber diffusion properties, and/or reducing the swelling of the fiber in water.

The present disclosure therefore provides a method for permanently reshaping keratin fibers, such as hair, comprising:

applying onto the keratin fibers a pre-treating composition comprising, in a cosmetically acceptable medium, at least one entity chosen from non reducing inorganic acids, non reducing organic acids, and salts thereof, wherein the pH value of said pre-treating composition ranges from 2 to 5,

rinsing the keratin fibers, for example with water,
applying onto the keratin fibers a treating composition comprising, in a cosmetically acceptable medium, at least one hydroxide compound, and

reshaping the keratin fibers, wherein the reshaping occurs after applying the pre-treating composition and at any time before, during, or after applying the treating composition.

The disclosed method therefore comprises first applying a pre-treating composition.

As previously explained, the pre-treating composition comprises at least one entity chosen from non reducing inorganic acids, non reducing organic acids, and salts thereof.

The at least one non reducing inorganic acid present in the pre-treating composition may be selected from mono- and polyacids.

Typically, the at least one non reducing inorganic acid is selected from hydrochloric acid, phosphoric acid, sulfuric acid, and boric acid.

The at least one non reducing organic acid present in the pre-treating composition typically comprises at least one functional group chosen from carboxylic, sulfonic, phosphonic, and phosphoric acid functional groups. They may comprise other chemical functional groups, such as hydroxy or amino functional groups. They may be saturated or unsaturated.

The at least one non reducing monocarboxylic acid is typically selected from hydroxylated, non-hydroxylated, aliphatic, and aromatic acids. Suitable examples thereof include for example acetic acid, trichloroacetic acid, propanoic acid, butanoic acid, phenyl acetic acid, salicylic acid, benzylic acid, glycolic acid, lactic acid, ascorbic acid, salicylic acid derivatives such as 2-hydroxy-5-octanoyl-benzoic acid and 2-hydroxy-4-trimethylsilanylmethoxy benzoic acid, the jasmonic acid derivative, 3-hydroxy-2-pentyl-cyclopentyl acetic acid, pyruvic acid, and mandelic acid.

The at least one non reducing polycarboxylic acid is typically selected from aspartic acid, glutamic acid, oxalic acid, succinic acid, tartaric acid, mucic acid, citric acid, malic acid, maleic acid, phthalic acid, poly(ethylene glycol) bis(carboxymethyl)ethers, acrylic polyacid, copolymer of acrylic acid and maleic acid, polyaspartic acid, and carboxylic polydimethylsiloxanes.

The at least one non reducing acrylic polyacid used can have a molecular weight of 2,000 g/mol. The at least one copolymer of acrylic acid and maleic acid used typically has a molecular weight of 3,000 g/mol. The at least one polyaspartic acid used typically has a molecular weight of 3,000 g/mol. The at least one carboxylic polydimethylsiloxane used has typically a molecular weight of 920 g/mol or of 2330 g/mol. The at least one poly(ethylene glycol) bis(carboxymethyl)ether used has typically a molecular weight of 250 g/mol or of 600 g/mol.

The at least one non reducing sulfonic acid may be selected from benzene sulfonic acid, sulfonic acid HSO₂OH, taurine (2-aminoethane sulfonic acid), 2-[4-(2-hydroxy-ethyl)-piperazin-1-yl]ethane sulfonic acid (or HEPES), and (3E)-3-(4-{(E)-[7,7-dimethyl-3-oxo-4-(sulfomethyl)bicyclo[2.2.1]hept-2-ylidene]methyl}benzylidene)-7,7-dimethyl-2-oxobicyclo[2.2.1]hept-1-yl]methane sulfonic acid, the latter compound being marketed by the Chimex company under the trade name MEXORYL SX. MEXORYL SX has the following chemical formula:

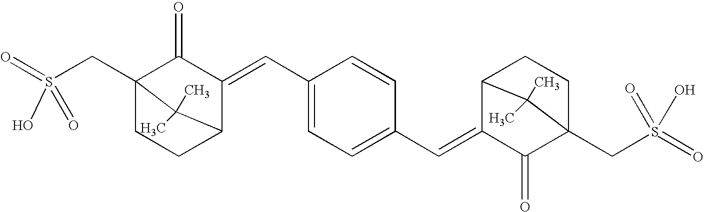

The at least one non reducing organic acid used in the pre-treating composition is for example a weak acid, defined herein as an acid which does not fully dissociate in water and which has a pKa value higher than 0. Among such weak acids, mention may be made of citric acid, acetic acid, salicylic acid, glycolic acid, trichloroacetic acid, L-glutamic acid, lactic acid, succinic acid, DL tartaric acid, poly(ethylene glycol) bis(carboxymethyl)ether having a molecular weight of 250 g/mol, salicylic acid derivatives such as 2-hydroxy-5-octanoyl-benzoic acid and 2-hydroxy-4-trimethylsilanylmethoxy-benzoic acid, the jasmonic acid derivative, 3-hydroxy-2-pentyl-cyclopentyl acetic acid, 2-[4-(2-hydroxy-ethyl)-piperazin-1-yl]-ethanesulfonic acid, malic acid, pyruvic acid, and mandelic acid.

Among the non reducing organic acids frequently used in the pre-treating composition mention may be made of citric acid, acetic acid, salicylic acid, and glycolic acid.

The pre-treating composition may in addition contain a plurality of acids and/or salts thereof as previously defined.

The pH value of the pre-treating composition ranges from 2 to 5, such as from 2.5 to 4.

For this purpose, the pH value is typically stabilized via a buffer solution.

As used herein, a “buffer solution” means a solution which pH value does not vary upon dilution or when adding a base or an acid in small amount.

The formulation of buffer solutions is well known to one skilled in the art.

When the pre-treating composition is applied onto the keratin fibers, its temperature ranges, for example, from 25 to 60° C., such as from 35 to 50° C.

Typically, the pre-treating composition is allowed to stand for a period of exposure time ranging from 10 to 60 minutes, such as from 10 to 40 minutes.

After having applied the pre-treating composition and when the exposure time is over, if any, the keratin fibers are rinsed off, for example with water.

In at least one embodiment of the present disclosure, after rinsing, and before the treating composition is applied, the disclosed method further comprises “wringing” the keratin fibers, for example squeezing or towel-drying with two towels. The keratin fibers will frequently not be fully dried.

As previously explained, the method of the disclosure comprises, after applying the pre-treating composition, applying onto the keratin fibers a treating composition comprising, in a cosmetically acceptable medium, at least one hydroxide compound.

As used herein, a “hydroxide compound” is defined as a compound that may release hydroxide ions.

For example, all the hydroxide compounds traditionally used in the lanthionization methods may be employed in the treating composition used according to the disclosure.

The at least one hydroxide compound present in the treating composition used according to the disclosure is for example selected from alkaline metal hydroxides, alkaline earth metal hydroxides, transition metal hydroxides, lanthanide metal hydroxides, actinide metal hydroxides, Group III hydroxides, Group IV hydroxides, Group V hydroxides, Group VI hydroxides, organic hydroxides, and compounds comprising at least one partially hydrolyzable, hydroxide substituent.

Examples of the at least one hydroxide compound to be suitably used in the present method include for example sodium hydroxide, guanidinium hydroxide, lithium hydroxide, calcium hydroxide, barium hydroxide, potassium hydroxide, magnesium hydroxide, aluminium hydroxide, copper hydroxide, and zinc hydroxide.

In at least one embodiment, sodium hydroxide and guanidinium hydroxide are for example, the at least one hydroxide compound herein.

The treating composition used in the disclosed method typically is in the form of an emulsion, such as an oil-in-water or a water-in-oil emulsion.

When the treating composition used according to the disclosure is in the form of an emulsion, it typically comprises at least one emulsifying agent chosen from non ionic, anionic, cationic, and amphoteric emulsifying agents.

The at least one emulsifying agent is selected depending on the emulsion that should be provided, i.e., a water-in-oil emulsion (W/O) or an oil-in-water emulsion (O/W).

For oil-in-water emulsions (O/W), examples of emulsifying agents to be suitably used include the following ones:

as amphoteric emulsifying agents, N-acyl-aminoacids such as N-alkylaminoacetates and disodium cocoamphodiacetate and amine oxides such as stearamine oxide;
as anionic emulsifying agents, acylglutamates such as “disodium hydrogenated tallow glutamate” (AMISOFT HS-21® marketed by the Ajinomoto company); carboxylic acids and their salts, such as sodium stearate; phosphoric esters and their salts, such as “DEA oleth-10 phosphate”; sulfosuccinates such as “disodium PEG-5 citrate lauryl sulfosuccinate” and “disodium ricinoleamido MEA sulfosuccinate”;
as cationic emulsifying agents, alkyl imidazolidinium, such as isostearyl ethylimidonium ethosulfate; ammonium salts, such as N,N,N-trimethyl-1-docosanaminium chloride (behentrimonium chloride);
as non ionic emulsifying agents, ose-type esters and ethers, such as sucrose stearate, sucrose cocoate and a combination of sorbitan stearate and sucrose cocoate marketed by the ICI company under the trade name ARLATONE 2121®; polyol esters, for instance glycerol or sorbitol esters, such as glyceryl stearate, polyglyceryl-2 stearate, sorbitan stearate; glycerol ethers; oxyethylene and/or oxypropylene ethers, such as oxyethylene or oxypropylene ether of lauryl alcohol comprising 25 oxyethylene groups and 25 oxypropylene groups (CTFA name “PPG-25 laureth-25”) and oxyethylene ether from the mixture of C₁₂-C₁₅ fatty alcohols comprising 7 oxyethylene groups (CTFA name “C₁₂-C₁₅ Pareth-7”); ethylene glycol polymers, such as PEG-100, and combinations thereof.

At least one of these emulsifying agents may be used.

For water-in-oil emulsions (W/O), suitable examples of emulsifying agents include polyol fatty esters, such as glycerol or sorbitol fatty esters, and for example polyol isostearic, oleic and ricinoleic esters, such as the mixture composed of petrolatum, polyglyceryl-3 oleate, glyceryl isostearate, hydrogenated castor oil and ozokerite, sold under the trade name PROTEGIN W® by the Goldschmidt company, sorbitan isostearate, polyglyceryl di-isostearate, polyglyceryl-2 sesqui-isostearate; ose-type esters and ethers such as “methyl glucose dioleate”; fatty esters, such as magnesium lanolate; dimethicone copolyols and alkyl-dimethicone copolyols, such as laurylmethicone copolyol sold under the trade name DOW CORNING 5200 FORMULATION AID by the Dow Corning company, cetyl dimethicone copolyol sold under the trade name ABIL EM 90® by the Goldschmidt company, and dimethicone copolyol sold under the trade name KF-6015 by the Shin-Etsu company; and combinations thereof.

The at least one hydroxide compound is typically present in the treating composition in an amount ranging from 0.2 to 1M, such as from 0.4 to 0.6M.

When the treating composition is applied onto the keratin fibers, its temperature typically ranges from 20 to 40° C., such as from 25 to 35° C.

The treating composition is typically allowed to react for a time period that is sufficient to allow hair reshaping. The treating composition is typically left for a period of exposure time ranging from 5 to 60 minutes, such as for 10 to 20 minutes.

The lanthionization is completed when the expected level of hair reshaping is obtained.

After the treating composition application, and after the optional exposure time, the keratin fibers are typically rinsed off, for example with water.

In at least one embodiment of the present disclosure, the disclosed method further comprises applying onto the keratin fibers a post-treating composition comprising, in a cosmetically acceptable medium, at least one entity selected from inorganic acids, organic acids, and salts thereof.

The at least one organic acid present in the post-treating composition is typically selected from acids comprising at least one functional group selected from carboxylic, sulfonic, phosphonic, and phosphoric acid functional groups. They may comprise other chemical functional groups, such as hydroxy or amino functions. They may be saturated or unsaturated.

By way of example of organic acids, mention may be made of acetic acid, lactic acid, glycolic acid, ascorbic acid, malic acid, benzylic acid, succinic acid, taurine, tartaric acid, and citric acid.

Citric acid is a frequently used acid.

The at least one inorganic acid present in the post-treating composition is typically selected from mono- and polyacids.

By way of example of inorganic acids, mention may be made of hydrochloric acid, orthophosphoric acid, sulfuric acid, and boric acid.

The post-treating composition typically has a pH value ranging from 2 to 7, such as from 2.5 to 4. The pH value of the composition may be adjusted via at least one alkaline agent selected from ammonia, monoethanol amine, diethanol amine, triethanol amine, propanediamine-1,3, ammonium carbonate or bicarbonate, an organic carbonate and an alkaline hydroxide. The composition is typically applied at room temperature for a period of exposure time ranging from 1 to 20 minutes, such as for 3 to 10 minutes.

As previously explained, the at least one entity chosen from non reducing inorganic acids, non reducing organic acids, and salts thereof present in the pre-treating composition, the at least one hydroxide compound present in the treating composition, and the at least one entity selected from inorganic acids, organic acids, and salts thereof present in the post-treating composition are contained in a cosmetically acceptable medium.

The cosmetically acceptable medium of the pre-treating composition, the treating composition, and the post-treating composition are typically independently selected from water, C₁-C₆ alcohols, for example alkanols such as ethanol, propanol, and isopropanol, alkane diols such as ethylene glycol, propylene glycol and pentanediol, benzyl alcohol, C₅-C₁₀ alkanes, acetone, methylethylcetone, C₂-C₆ esters, such as methyl acetate, butyl acetate and ethyl acetate, dimethoxyethane, diethoxyethane, N-methylpyrrolidone (NMP), dimethylsulfoxide (DMSO), and combinations thereof.

The pre-treating composition, the treating composition, and the post-treating composition may in addition comprise traditional cosmetic additives selected for example from thickeners, softeners, antifoaming agents, sunscreen agents, moisturizing agents, dyes, pigments, fragrances, preserving agents, anionic, cationic, non ionic or amphoteric surfactants, non fixing polymers, volatile or non volatile silicones, mineral, vegetable, animal, or synthetic oils, proteins, vitamins, polyols, and combinations thereof.

In at least one embodiment of the disclosure, the keratin fibers undergo a heat treatment via a heating iron at a temperature ranging from 60 to 220° C., such as from 120 to 220° C., this heat treatment occurring during or after the application of the treating composition.

The present disclosure also provides a cosmetic kit for permanently reshaping the hair comprising a first compartment containing a pre-treating composition comprising, in a cosmetically acceptable medium, at least one entity chosen from non reducing inorganic acids, non reducing organic acids, and salts thereof, wherein the pH value of said pre-treating composition ranges from 2 to 5 and a second compartment containing a treating composition comprising, in a cosmetically acceptable medium, at least one hydroxide compound. If needed, the kit may optionally comprise a third compartment containing a post-treating composition comprising, in a cosmetically acceptable medium, at least one entity selected from inorganic acids, organic acids, and salts thereof.

The present disclosure is further illustrated by the following examples without being limiting in nature.

### EXAMPLES

### Example 1

1. Natural African Frizzy hair was straightened by carrying out the disclosed method.

The pre-treating composition used was a Titrisol buffer solution (pH 3) comprising 0.04 mol/L citric acid, 0.06 mol/L hydrochloric acid, and 0.08 mol/L soda.

The hair was dipped for 30 minutes into a bath at 40° C. filled with the pre-treating composition, with a bath ratio of 10 mL of pre-treating composition for one gram of hair.

The hair was then rinsed off with water.

The hair was towel-dried between 2 towels.

Thereafter the hair underwent straightening.

The hair straightening was effected using a 0.6M soda treating composition (pH value=14 before treatment) for 20 minutes at 30° C. Such straightening was characterized as follows.

The hair was dipped into the soda-containing composition, with a bath ratio of 40 ml/g, i.e. 10 ml per small lock of hair.

The hair was stretched for 4 minutes, then allowed to remain relaxed for 6 minutes.

The lock of hair was then turned over and the operation was repeated: 4 minutes stretching and 6 minutes relaxing.

The hair was then rinsed off by being dipped into water.

Moisture was then squeezed from the hair between two towels.

The hair was dipped into a citric acid bath pH 3 for 5 minutes at room temperature, with a bath ratio of about 50 ml/g.

The hair was then rinsed off with water and washed with shampoo, and at last let air dry.

2. Determining the impact of the pre-treating composition on the reshaping properties.

The straightening performance was determined qualitatively.

A comparison was made between the reshaping of hair treated using the method of the disclosure described under 1 above, and control hair with no reshaping, and hair that was reshaped using the method described under 1 but without applying the pre-treating composition.

The resulting observations demonstrated that the pre-treatment using an acid-containing pre-treating composition did not affect the reshaping performance of a soda-based straightening method.

### Example 2

1. Determining the hair quality improvement resulting from the disclosed method. The swelling of the hair in water was considered.

All the treatments were conducted on small locks of African hair weighing 250 mg.

A first hair sample (sample no. °1) was reshaped by carrying out the method of the disclosure. The hair therefore underwent the following steps:

an acetic acid-containing pre-treating composition, pH 3, at 40° C. was applied onto the hair and left for 30 minutes; with a bath ratio of 10 ml/g;

the hair was rinsed with running water;

hair moisture was removed by squeezing using two towels;

hair straightening.

The hair straightening was effected using a 0.6M soda treating composition (pH value=14 before treatment) for 20 minutes at 30° C. Such straightening was characterized as follows.

The hair was dipped into the soda-containing composition, with a bath ratio of 40 ml/g, i.e. 10 ml per small lock of hair.

The hair was stretched for 4 minutes, then allowed to remain relaxed for 6 minutes.

The lock of hair was then turned over, and the operation was repeated: 4 minutes stretching and 6 minutes relaxing.

The hair was then rinsed off by being dipped into water.

Moisture was then squeezed from the hair between two towels.

The hair was dipped into a citric acid bath pH 3 for 5 minutes at room temperature, with a bath ratio of about 50 ml/g.

The hair was then rinsed off with water and washed with shampoo, and at last let air dry.

A further sample (control sample) was straightened using the hereabove defined treating composition, but without applying the acid-containing pre-treating composition (and therefore no rinsing, no towel-drying).

A further sample (sample no. °2) was treated according to a method comprising a pre-treating step, a towel-drying step, and a straightening step, such as defined hereabove, but with no rinsing step.

A further sample (sample no. °3) was treated according to a method comprising pre-treating such as defined hereabove, rinsing such as defined hereabove, drying using a hood hair dryer at 60° C. for 10 minutes, and soda-based straightening such as previously defined.

2. Swelling measurements were collected by an optical system Zimmer 3D.

The hair to be tested was introduced attached to a weight in a quartz vessel filled with distilled water at 25° C.

The hair average diameter was evaluated by averaging the 250 adjacent measures effected either in a dry environment or in water. This measuring lasted for about 10 minutes.

The average swelling, depicting the hair quality, was determined by the change in average diameter that occurred between the dry condition and the dipped condition.

3. The results were as follows.

The various treatments all resulted in a similar straightening level.

The difference in hair quality following these various treatments was evaluated via the water swelling test.

The results obtained are given in Table 1.

TABLE 1

| | | Dry state | Average | Average swelling | |
|---|---|---|---|---|---|
| | | average | diameter in | | Standard |
| | | diameter | water | | deviation |
| Sample | Treatment | (μm) | (μm) | Mean (%) | (%) |
| Control | No pre-treatment | 91.3 | 115.9 | 27.4 | 9.0 |
| 1 | Pre-treatment + rinsing + | 108.7 | 112.9 | 4.0 | 2.5 |
| | towel-drying + | | | | |
| | straightening | | | | |
| 2 | Pre-treatment + | 76.3 | 96.4 | 20.9 | 4.7 |
| | towel-drying + straightening | | | | |
| 3 | Pre-treatment + rinsing + | 85.9 | 107.3 | 24.9 | 2.4 |
| | drying + straightening | | | | |

The intermediate rinsing step had a positive influence on the post-straightening hair quality, as illustrated through a reduced swelling of the hair in water. Such effect was significant, provided that this rinsing step was followed with a towel-drying of the hair, rather than with a hood- or a air-dryer-drying, prior to conducting the straightening step.

### Example 3

### A. Determining the Hair Quality Improvement Resulting from the Method of the Disclosure. The Swelling of the Hair in Water was Considered

All the treatments were conducted on small locks of African hair weighting 250 mg.

The reshaping of two hair samples was effected by carrying out the disclosed method. Therefore, the hair underwent the method as described hereunder.

1. Acid-Based Pre-Treatment

A pre-treating composition was applied onto the hair, either based on glycolic acid, or on salicylic acid, or on citric acid.

The glycolic acid-based pre-treating composition comprised 23 mg of glycolic acid for 10 ml of water.

The salicylic acid-based pre-treating composition comprised 20 mg of salicylic acid for 10 ml of water.

The citric acid-based pre-treating composition comprised 2.7 mg of citric acid for 10 ml of water.

The pre-treating composition was applied onto the hair for 30 minutes at 40° C. in a beaker placed in a water bath, with a bath ratio of 10 ml/g, i.e. 2.5 ml of composition per small lock of hair.

2. Rinsing

The hair was rinsed off with water.

3. Towel-Driving.

Moisture was squeezed from the hair between two towels.

4. Straightening

The hair straightening was effected using a 0.6M soda treating composition (pH value=14 before treatment) for 20 minutes at 30° C. Such straightening was characterized as follows.

The hair was dipped into the soda-containing composition, with a bath ratio of 40 ml/g, i.e. 10 ml per small lock of hair.

The hair was stretched for 4 minutes, then allowed to remain relaxed for 6 minutes.

The lock of hair was then turned over, and the operation was repeated: 4 minutes stretching and 6 minutes relaxing.

The hair was then rinsed off by being dipped into water.

Moisture was then squeezed from the hair between two towels.

5. Citric Acid Neutralization

The hair was dipped into a citric acid bath pH 3 for 5 minutes at room temperature, with a bath ratio of about 50 mug. The hair was then rinsed off with water and washed with shampoo, and at last let air dry.

### B. Determining the Swelling of the Treated Hair in Water

Swelling measurements were collected by an optical system Zimmer 3D to follow the change in the hair diameter when dipped into water. The hair to be tested was introduced attached to a weight in a quartz vessel filled with distilled water at 25° C. The diameter changes were then continuously read for 5 minutes. Beyond 5 minutes, the diameter value was stabilized.

Based upon these figures, the average swelling of the fiber could be calculated for each treatment.

The two measurement methods using the Zimmer 3D (example 2) and Zimmer (example 3) are not the same, but the information they provide are considered similar. With the ZIMMER 3D, one should wait a few minutes, then the hair diameter can be read after stabilization. The stabilized diameter value was given. With the ZIMMER, the change in the hair diameter was read from 0 to 5 minutes, and beyond 5 minutes, the diameter stabilized value was given.

The results correspond to the average of 15 tests for each treatment.

The control hair, having been treated according to the method as described hereabove except the acid-based pre-treating step was lacking, had a swelling in water of 50.43%.

The hair that had been treated according to the method as described hereabove and the acid-based pre-treatment of which was effected using either the citric acid-containing composition or the glycolic acid-containing composition had a much lower swelling in water as compared to the hair with no pre-treatment.

The method of the disclosure thus had a positive influence on the hair quality after straightening, illustrated through a reduced swelling of the hair in water.

What is claimed is:

1. A method for permanently reshaping keratin fibers, comprising:
applying onto the keratin fibers a pre-treating composition comprising, in a cosmetically acceptable medium, at least one entity chosen from non reducing inorganic acids, non reducing organic acids, and salts thereof, wherein the pH value of said pre-treating composition ranges from 2 to 5,
rinsing the keratin fibers,
applying onto the keratin fibers a treating composition comprising, in a cosmetically acceptable medium, at least one hydroxide compound, and
reshaping the keratin fibers, wherein the reshaping occurs after applying the pre-treating composition and at any time before, during, or after applying the treating composition.

2. A method according to claim 1, wherein the at least one non reducing inorganic acid is selected from mono- and polyacids.

3. A method according to claim 2, wherein the at least one non reducing inorganic acid is selected from hydrochloric acid, phosphoric acid, sulfuric acid, and boric acid.

4. A method according to claim 1, wherein the at least one non reducing organic acid comprises at least one functional group chosen from carboxylic, sulfonic, phosphonic, and phosphoric acid functional groups.

5. A method according to claim 4, wherein the at least one non reducing organic acid is a monocarboxylic acid selected from hydroxylated, non-hydroxylated, aliphatic, and aromatic acids.

6. A method according to claim 5, wherein the at least one non reducing monocarboxylic, organic acid is selected from acetic acid, trichloroacetic acid, propanoic acid, butanoic acid, phenyl acetic acid, salicylic acid, benzylic acid, glycolic acid, lactic acid, ascorbic acid, salicylic acid derivatives, jasmonic acid derivative, 3-hydroxy-2-pentyl-cyclopentyl acetic acid, pyruvic acid, and mandelic acid.

7. A method according to claim 4, wherein the at least one non reducing organic acid is a polycarboxylic acid selected from aspartic acid, glutamic acid, oxalic acid, succinic acid, tartaric acid, mucic acid, citric acid, malic acid, maleic acid, phthalic acid, poly(ethylene glycol) bis(carboxymethyl)ethers, acrylic polyacid, copolymer of acrylic acid and maleic acid, polyaspartic acid, and carboxylic polydimethylsiloxanes.

8. A method according to claim 4, wherein the at least one non reducing organic acid is a sulfonic acid selected from benzene sulfonic acid, sulfonic acid HSO₂OH, taurine, 2-[4-(2-hydroxy-ethyl)-piperazin-1-yl]ethane sulfonic acid (or HEPES), and (3E)-3-(4-{(E)-[7,7-dimethyl-3-oxo-4-(sulfomethyl)bicyclo[2.2.1]hept-2-ylidene]methyl}benzylidene)-7,7-dimethyl-2-oxobicyclo[2.2.1]hept-1-yl]methane sulfonic acid.

9. A method according to claim 1, wherein the at least one non reducing organic acid is selected from trichloroacetic acid, L-glutamic acid, lactic acid, succinic acid, DL tartaric acid, poly(ethylene glycol) bis(carboxymethyl)ether having a molecular weight of 250 g/mol, salicylic acid derivatives, jasmonic acid derivative, 3-hydroxy-2-pentyl-cyclopentyl acetic acid, 2-[4-(2-hydroxy-ethyl)-piperazin-1-yl]-ethanesulfonic acid, malic acid, pyruvic acid, and mandelic acid.

10. A method according to claim 1, wherein the at least one non reducing organic acid is selected from citric acid, acetic acid, salicylic acid, and glycolic acid.

11. A method according to claim 1, wherein the pre-treating composition is at a temperature ranging from 25 to 60° C.

12. A method according to claim 1, wherein the pre-treating composition is allowed to stand for a period of exposure time ranging from 10 to 60 minutes.

13. A method according to claim 1, further comprising wringing the keratin fibers, wherein the wringing occurs after the fibers are rinsed and before the treating composition is applied.

14. A method according to claim 1, wherein the at least one hydroxide compound is selected from alkaline metal hydroxides, alkaline earth metal hydroxides, transition metal hydroxides, lanthanide metal hydroxides, actinide metal hydroxides, Group III hydroxides, Group IV hydroxides, Group V hydroxides, Group VI hydroxides, organic hydroxides, and compounds comprising at least one partially hydrolyzable, hydroxide substituent.

15. A method according to claim 14, wherein the at least one hydroxide compound is selected from sodium hydroxide, guanidinium hydroxide, lithium hydroxide, calcium hydroxide, barium hydroxide, potassium hydroxide, magnesium hydroxide, aluminium hydroxide, copper hydroxide, and zinc hydroxide.

16. A method according to claim 15, wherein the at least one hydroxide compound is selected from sodium hydroxide and guanidinium hydroxide.

17. A method according to claim 1, wherein the treating composition is in the form of an emulsion.

18. A method according to claim 1, wherein the at least one hydroxide compound is present in the treating composition in an amount ranging from 0.2 to 1M.

19. A method according to claim 1, wherein the treating composition is at a temperature ranging from 20 to 40° C.

20. A method according to claim 1, wherein the treating composition is allowed to react for a period of exposure time ranging from 5 to 60 minutes.

21. A method according to claim 1, wherein the keratin fibers are rinsed off after the treating composition is applied.

22. A method according to claim 1, further comprising applying onto the keratin fibers a post-treating composition comprising, in a cosmetically acceptable medium, at least one entity selected from inorganic acids, organic acids, and salts thereof.

23. A method according to claim 22, wherein the at least one organic acid present in the post-treating composition is selected from saturated and unsaturated acids comprising at least one functional group selected from carboxylic, sulfonic, phosphonic, and phosphoric acid functional groups.

24. A method according to claim 23, wherein the at least one organic acid present in the post-treating composition is selected from acetic acid, lactic acid, glycolic acid, ascorbic acid, malic acid, benzylic acid, succinic acid, taurine, tartaric acid, and citric acid.

25. A method according to claim 22, wherein the at least one inorganic acid is selected from mono- and polyacids.

26. A method according to claim 25, wherein the at least one inorganic acid is selected from hydrochloric acid, phosphoric acid, sulfuric acid, and boric acid.

27. A cosmetic kit for permanently reshaping the hair comprising:
a first compartment containing a pre-treating composition comprising, in a cosmetically acceptable medium, at least one entity chosen from non reducing inorganic acids, non reducing organic acids, and salts thereof, wherein the pH value of said pre-treating composition ranges from 2 to 5,
a second compartment containing a treating composition comprising, in a cosmetically acceptable medium, at least one hydroxide compound, and
optionally, a third compartment containing a post-treating composition comprising, in a cosmetically acceptable medium, at least one entity selected from inorganic acids, organic acids, and salts thereof.
